(19) United States
(12) Patent Application Publication
(10) Pub. No.: US 2009/0022784 A1
(43) Pub. Date: Jan. 22, 2009

(54) SYSTEMS, DEVICES, AND METHODS FOR IONTOPHORETIC DELIVERY OF COMPOSITIONS INCLUDING LIPOSOME-ENCAPSULATED INSULIN

(76) Inventors: Kentaro Kogure, Sapporo (JP); Misuzu Watanabe, Sapporo (JP); Hideyoshi Harashima, Sapporo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/138,230

(22) Filed: Jun. 12, 2008

Related U.S. Application Data
(60) Provisional application No. 60/983,071, filed on Oct. 26, 2007.

(30) Foreign Application Priority Data
Jun. 12, 2007 (JP) 2007-155530

(51) Int. Cl. A61K 9/127; A61K 38/28; A61P 3/00

(52) U.S. Cl. 424/450; 514/3

(57) ABSTRACT

Systems, devices, and methods for delivering one or more active ingredients to intradermal tissues, deep regions of pores, and intradermal tissues in the vicinity of pores. In some embodiments, a composition is provided including a plurality of liposomes including a cationic lipid, and an amphiphilic glycerophospholipid having a saturated fatty acid moiety and an unsaturated fatty acid moiety, and at least one insulin, insulin analog, or insulin derivative.

### CROSS-REFERENCE AND RELATED APPLICATIONS

This application claims the benefit under 35 U.S.C. § 119(e) of U.S. Provisional Patent Application No. 60/983,071 filed Oct. 26, 2007, the entire contents of which are incorporated herein by reference. This application also claims benefit of priority under 35 U.S.C. § 119 to Japanese Patent Application No. 2007-155530, filed Jun. 12, 2007.

### BACKGROUND

1. Technical Field

This disclosure generally relates to the field of transdermal administration of active ingredients by iontophoresis and, more particularly, to compositions useful for transdermally administering an insulin molecule via iontophoresis to, for example, regions of a pore and intradermal tissue around the pore. Compositions and methods described herein are particularly useful for preventing or treating diabetes.

2. Description of the Related Art

Diabetes mellitus is a metabolic disease that is characterized by and results from a blood glucose level that is high compared to that of a normal, healthy subject. The abnormally high blood glucose level is due to an inadequate amount of insulin or to an inability to use the insulin that is present in the body. The high blood glucose levels in diabetes leads to complications such as microangiopathy and arteriosclerosis in the kidney, retina, nerve, etc., resulting in life-threatening medical conditions. Various types of diabetes are known, in particular, diabetes mellitus type I and type II. Type I diabetes is caused by a deficiency of insulin secretion in the β-cells of the islets of Langerhans in the pancreas and thus a deficiency of circulating insulin. Type II diabetes results from a decrease in both insulin secretion and insulin sensitivity.

Diabetes can be treated by administering insulin to an individual diagnosed with the disease. Particularly useful methods for treating diabetes include continuous administration of insulin to an organism to maintain blood glucose level within a normal range. Such treatment is known to be effective for treatment of either type I or type II diabetes. Insulin can be administered, for example, by hypodermoclysis, that is, subcutaneously by either manual injection, as needed, or continuously using a pump. While continuous injection may be more effective and convenient, it nevertheless requires the insertion of a needle for extended periods of time, which may interfere with the quality of life of patients and may increase the possibility of infections. Accordingly, there remains a need for a method to stably, effectively, and conveniently administer insulin to maintain insulin and blood glucose levels in an individual in need thereof.

Iontophoresis employs an electromotive force and/or current to transfer an active agent (e.g., a charged substance, an ionized compound, an ionic drug, a therapeutic, a bioactive agent, and the like) to a biological interface (e.g., skin, mucous membrane, and the like) by applying an electrical potential to an electrode proximate an iontophoretic changer comprising a similarly charged active ingredient and/or its vehicle. For example, a positively charged ion is transferred into the skin at an anode side of an electric system of an iontophoresis device. In contrast, a negatively charged ion is transferred into the skin at a cathode side of the electric system of the iontophoresis device.

Although skin is one of the most extensive and readily accessible organs, it has historically been difficult to deliver certain active agents transdermally. Often a drug is administered to a living body mainly through the corneum of the skin. The corneum, however, is a lipid-soluble high-density layer that makes the transdermal administration of highly water-soluble substances and polymers, such as peptides, nucleic acids, and the like, difficult.

Commercial acceptance of transdermal delivery devices or pharmaceutically acceptable vehicles is dependent on a variety of factors including cost to manufacture, shelf life, stability during storage, efficiency and/or timeliness of active agent delivery, biological capability, and/or disposal issues. Commercial acceptance of transdermal delivery devices or pharmaceutically acceptable vehicles is also dependent on their versatility and ease-of-use.

The present disclosure is directed to overcoming one or more of the shortcomings set forth above, and/or providing further related advantages.

### BRIEF SUMMARY

This disclosure is directed to systems, devices and methods for delivering one or more active ingredients to intradermal tissues, deep regions of pores, and intradermal tissues in the vicinity of pores.

In some embodiments, the disclosed compositions and/or formulations include liposome-encapsulated insulin molecules that are stable and suitable for delivery to a skin pore and to the intradermal tissue surrounding deep portions of a skin pore. In some embodiments, the disclosed compositions and/or formulations can be iontophoretically delivered intradermally via a skin pore to the deep portions of the skin pore and to the surrounding tissue. In some embodiments, administration of the disclosed compositions and/or formulations can decrease a blood glucose level and continuously maintain the decreased blood glucose level for an extended period of time. In some embodiments, administration of the disclosed compositions and/or formulations provides for prevention or treatment of diabetes. In certain embodiments, needle injection of the disclosed compositions and/or formulations is unnecessary. Accordingly, in certain embodiments, delivery of insulin by methods disclosed herein may improve the quality of life of the recipient.

In some embodiments, a composition for iontophoretic delivery of at least one insulin molecule to a biological subject comprises: a plurality of liposomes comprising a cationic lipid and an amphiphilic glycerophospholipid having a saturated fatty acid moiety and an unsaturated fatty acid moiety; and at least one insulin molecule; wherein the at least one insulin molecule is enclosed within a liposome; and wherein the composition is suitable for iontophoretic delivery of the at least one insulin molecule to a biological subject. In some embodiments, the at least one insulin molecule is an insulin and/or an insulin analog and/or a derivative of an insulin and/or a derivative of an insulin analog. In some embodiments, the at least one insulin molecule is an ultra-fast-acting insulin analog and/or a long-acting insulin analog.

In some embodiments, a composition for iontophoretic delivery of at least one insulin molecule comprises a plurality of liposomes wherein the plurality of liposomes comprises a cationic lipid comprising a C1-20 alkane substituted with a C1-22 acyloxy group and a tri-C1-6 alkylammonium group. In some embodiments, the cationic lipid comprises a C1-20 alkane substituted with at least two C1-22 acyloxy groups and at least one tri-C1-6 alkylammonium group. In some embodiments, the cationic lipid comprises 1,2-dioleoyloxy-3-(trimethylammonium)propane.

In some embodiments, a composition for iontophoretic delivery of at least one insulin molecule comprises a plurality of liposomes wherein the plurality of liposomes comprises an amphiphilic glycerophospholipid comprising phosphatidylcholine or egg yolk phosphatidylcholine.

In some embodiments, a composition for iontophoretic delivery of at least one insulin molecule comprises a plurality of liposomes wherein the plurality of liposomes comprises an amphiphilic glycerophospholipid having a saturated fatty acid moiety wherein the saturated fatty acid moiety is a C12-22 saturated fatty acid. In some embodiments, the saturated fatty acid moiety is selected from palmitic acid, lauric acid, myristic acid, pentadecylic acid, margaric acid, stearic acid, tuberculostearic acid, arachidic acid, or behenic acid. In some embodiments, the saturated fatty acid moiety comprises 1, 2, 3, 4, 5 or 6 carbon-carbon unsaturated double bonds.

In some embodiments, a composition for iontophoretic delivery of at least one insulin molecule comprises a plurality of liposomes wherein the plurality of liposomes comprises an amphiphilic glycerophospholipid having an unsaturated fatty acid moiety wherein the unsaturated fatty acid moiety is a C14-22 unsaturated fatty acid. In some embodiments, the unsaturated fatty acid moiety is selected from oleic acid, myristoleic acid, palmitoleic acid, elaidic acid, vaccenic acid, gadoleic acid, erucic acid, nervonic acid, linoleic acid, α-linoleic acid, eleostearic acid, stearidonic acid, arachidonic acid, eicosapentaenoic acid, clupanodonic acid, or docosahexaenoic acid.

In some embodiments, a composition for iontophoretic delivery of at least one insulin molecule comprises a plurality of liposomes wherein the plurality of liposomes comprises a cationic lipid and an amphiphilic glycerophospholipid wherein a molar ratio of the cationic lipid to the amphiphilic glycerophospholipid is from about 9:1 to about 1:9. In some embodiments, the molar ratio is from about 3:2 to about 2:3.

In some embodiments, a composition for iontophoretic delivery of at least one insulin molecule comprises a plurality of liposomes wherein the plurality of liposomes comprises a cationic lipid and an amphiphilic glycerophospholipid and further comprises a sterol. In some embodiments, the sterol is selected from C₁₂₋₃₁ cholesteryl fatty acid, C₁₂₋₃₁ dihydrocholesteryl fatty acid, polyoxyethylene cholesteryl ether, or polyoxyethylene dihydrocholesteryl ether. In some embodiments, the sterol is selected from cholesterol, cholesteryl lanolate, cholesteryl oleate, cholesteryl nonanate, macadamia nut fatty acid cholesteryl, or polyoxyethylene dihydrocholesteryl ether. In some embodiments, the sterol is cholesterol. In some embodiments, a molar ratio of the cationic lipid to the sterol is from about 19:1 to about 1:1. In some embodiments, a molar ratio of the amphiphilic glycerophospholipid to the sterol is from about 19:1 to about 1:1. In some embodiments, a molar ratio of the cationic lipid to the total of the amphiphilic glycerophospholipid and the sterol is from about 9:1 to about 1:9. In some embodiments, a molar ratio of the cationic lipid to the amphiphilic glycerophospholipid to the sterol is about 4:4:1.

In some embodiments, a composition for iontophoretic delivery of at least one insulin molecule comprises a plurality of liposomes wherein an average particle diameter of the plurality of liposomes is about 400 nm or greater. In some embodiments, an average particle diameter of the plurality of liposomes is from about 400 nm to about 1000 nm.

In some embodiments, the disclosed compositions and/or formulations are sterile and are delivered in a sanitary manner.

In some embodiments, a method for treating or preventing a condition or a disease associated with increased blood glucose levels in a biological subject comprises iontophoretically administering to the biological subject in need of such treatment a therapeutically effective amount of a composition comprising a plurality of liposomes comprising a cationic lipid, an amphiphilic glycerophospholipid having a saturated fatty acid moiety and an unsaturated fatty acid moiety, and at least one insulin molecule, the at least one insulin molecule being carried by the plurality of liposomes. In some embodiments of the method, the cationic lipid is present in a molar ratio of the cationic lipid to the amphiphilic glycerophospholipid of about 9:1 to about 1:9. In some embodiments of the method, the liposome has a mean particle diameter of about 400 nm to about 1000 nm. In some embodiments of the method, iontophoretically administering the therapeutically effective amount of a composition comprises providing a current ranging from about 0.1 mA/cm2 to about 0.6 mA/cm2, or from about 0.3 mA/cm2 to about 0.5 mA/cm2, or about 0.45 mA/cm2, for a pre-selected period of time. In some embodiments, the condition or disease associated with increased blood glucose levels is diabetes mellitus or, in particular, diabetes mellitus type 1.

In some embodiments, a composition, formulation or method is provided for controlled or sustained release of an insulin molecule into the circulation of a biological subject. In some embodiments, release of a liposome enclosing an insulin molecule into the circulation from pores or intradermal tissue in the vicinity of pores is controlled or sustained. In some embodiments, release of an insulin molecule from a liposome into the circulation is controlled or sustained.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is a schematic diagram of an iontophoresis device used for administration of a liposome in performing an in vivo skin penetration test according to one illustrated embodiment.

FIG. 2 is a plot of changes over time of blood glucose levels of rats after administration of an insulin-containing liposome preparation according to one described embodiment.

FIG. 3 is a flow diagram of a method for treating or preventing a condition or disease associated with increased blood glucose levels in a biological subject according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with delivery devices including, but not limited to, voltage and/or current regulators, or protective coverings and/or liners to protect delivery devices during shipping and storage, have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment,” or “in some embodiments” means that a particular referent feature, structure, or characteristic described in connection with the embodiment is included in at least one embodiment. Thus, the appearance of the phrases “in one embodiment,” or “in an embodiment,” or “in another embodiment,” or “in some embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an iontophoretic delivery liposome formulation including an insulin molecule includes a single species of insulin molecule, and may also include two or more species of insulin molecules, including analogs and/or derivatives thereof. It should also be noted that the term “or” is generally employed in its sense including “and/or” unless the content clearly dictates otherwise.

Unless otherwise specified, the variable “Cₙ” in a group, or as part of a group, generally refers to the “total number of carbon atoms n” in the group or the part of a group. Thus, for example, “C₁₋₆ saturated fatty acid” refers to a “saturated fatty acid containing from 1 to 6 carbon atoms”, and “C₁₂₋₃₁ cholesteryl fatty acid ester” refers to a “cholesteryl fatty acid ester containing from 12 to 31 carbon atoms”.

The terms “alkyl”, “alkenyl”, or “alkynyl” as a group or as part of a group generally refer to, unless otherwise specified, straight chain, branched chain, cyclic, substituted, or unsubstituted hydrocarbon radicals. In some embodiments, the “alkyl”, “alkenyl”, or “alkynyl” are selected from the group consisting of straight chain alkyls, alkenyls, or alkynyls and branched chain alkyls, alkenyls, or alkynyls. In some embodiments, the “alkyl”, “alkenyl”, or “alkynyl” is selected from the group consisting of straight chain alkyls, alkenyls, and alkynyls.

The term “aryl” generally refers to, unless otherwise specified, aromatic monocyclic or multicyclic hydrocarbon ring system consisting only of hydrogen and carbon and containing from 6 to 19 carbon atoms, where the ring system may be partially or fully saturated. Aryl groups include, but are not limited to, groups such as phenyl and naphthyl.

The term “heteroaryl” generally refers to, unless otherwise specified, a 5- to 6-membered partially or fully aromatic ring radical which consists of one to three heteroatoms selected from the group consisting of nitrogen, oxygen, and sulfur.

It is understood that, in use of the terms “fatty acid cholesteryl” or “fatty acid dihydrocholesteryl” herein, the fatty acid may be a saturated or an unsaturated fatty acid. Further, the fatty acid may be a straight-chain, branched, or cyclic fatty acid. In certain embodiments, the fatty acid in the “fatty acid cholesteryl” is a straight-chain fatty acid. In certain embodiments, the fatty acid in the “fatty acid dihydrocholesteryl” is a straight-chain fatty acid.

The term “insulin molecule” generally refers to, unless otherwise specified, insulin, an insulin analog, a derivative of insulin or insulin analog, or any combination thereof. The term “insulin” includes any natural insulin extracted from a mammal, such as a human, a cow, or a pig, and any insulin prepared by recombinant technology, synthesis, semi-synthesis, or partial synthesis. The term “insulin analog” generally refers to, unless otherwise specified, a peptide having an insulin amino acid sequence with substitution of one or more amino acid residues and/or with addition or deletion of one or more amino acid residues. The term “derivative of insulin or insulin analog” refers to, unless otherwise specified, insulin or insulin analog wherein one or more amino acid residues is bonded to at least one organic substituent or is otherwise chemically modified.

The phrase “the insulin molecule is biologically active” means that an insulin molecule, when administered to an organism that is responsive to insulin, causes a decrease in the blood glucose level of the organism. Biological activity of an insulin molecule can be readily determined by one of skill in the relevant art. For example, glucose levels can be measured in blood, plasma or serum, using glucose sensors, glucose meters, glucose monitors, and the like, and/or according to established laboratory protocols.

The term “front surface” generally refers, unless otherwise specified, to a side near the skin of a living body on the path of electric current flowing through the inside of the electrode structure in administering liposomes.

The term “living body” generally includes mammals such as, for example, humans, rats, guinea pigs, rabbits, mice, dogs, cats, and pigs.

Iontophoretic delivery of active ingredients (e.g., insulin molecules, such as insulin, insulin analogs, derivatives of insulin or insulin analogs, and the like) may provide a way of avoiding the first-pass effect of the liver and may permit for easier control of initiation, cessation, etc., associated with the administration of a drug.

Although it may be possible to transdermally administer substances with various physicochemical properties using charged liposomes as carriers (see, e.g., Median, V. M., et al., Intl. J. Pharmaceutics 306(1-2): 1-14 (Dec. 8, 2005). Epub Nov. 2, 2005 Epub 2005 Nov. 2), the large particle diameter of liposomes often makes it difficult to pass through the corneum.

Pores, which pass from the skin surface to a deep region of the skin, may provide a route for transdermally administering liposomes efficiently (see, e.g., Hoffman, R. T., et al., Nat. Med. 1(7): 705-706 (July, 1995); Fleisher, D., et al., Life Sci. 57(13):1293-1297 (1995). It may be possible to, for example, administer liposomes enclosing an enzyme to hair follicle stem cells in pores by iontophoresis (see, e.g., Protopapa, E. E., et al., J. Eur. Acad. Dermatol. Venereol. 13(1):28-35 (July, 1999). It may also be possible to, for example, administer liposomes enclosing 5-aminolevulinic acid serving as an agent for a photodynamic therapy to pore sebaceous glands and the like in upper regions or pores by iontophoresis (see, e.g., Han, I., et al., Arch. Dermatol. Res. 295(5):210-217 (November, 2005); Epub 2005 Nov. 11). Han, I., et al., have also reported that liposomes enclosing adriamycin serving as a therapeutic agent pore-associated tumors may be delivered to pores by iontophoresis (Han, I., et al., Exp. Dermatol. 13(2):86-92 (February, 2004)).

Often in iontophoresis a drug is administered to surface portions of skin tissue, for example, to treat diseases of the surface of the skin, or the like. In some embodiments, a drug (e.g., insulin molecules, such as insulin, insulin analogs, derivatives of insulin or insulin analogs, and the like) is delivered systemically to a general circulatory system by means of subcutaneous blood vessels present in deep regions of a skin. In some embodiments, in which the goal is to efficiently treat or prevent diabetes, an agent such as insulin may be delivered to the intradermal tissues in the vicinity of the pores. Administration of insulin by conventional iontophoresis technology displays certain limitations. In particular, it is difficult to consistently maintain normal levels of blood glucose over an extended period of time, since with such methods the blood glucose is only temporarily lowered and then rapidly increases again (see, e.g., Kalia, Y. N., et al., Iontophoretic drug delivery. Advanced Drug Delivery Reviews 56:619-658 (2004)). Accordingly, an object of the compositions and methods for iontophoresis disclosed herein is to stably and efficiently deliver liposomes enclosing insulin molecules, such as insulin, or analogs or derivatives thereof, to deep regions of pores and intradermal tissues in the vicinity of pores to maintain blood glucose at normal levels for an extended period of time.

### Liposome Composition for Iontophoresis

As described above, in some embodiments, the disclosed composition includes an active ingredient (e.g., insulin molecules, such as insulin, insulin analogs, derivatives of insulin or insulin analogs, and the like) carried in a liposome, in which the liposome includes, as a constituent component, a cationic lipid, and an amphiphilic glycerophospholipid including both saturated fatty acid and unsaturated fatty acid moieties. It is an unexpected fact that liposomes comprising such specific constituent components advantageously provide stable delivery of one or more insulin molecules to deep regions of a pore and/or intradermal tissues in the vicinity of the pore by iontophoresis.

In some embodiments, a composition is provided for administering an active ingredient through a pore and/or intradermal tissues in the vicinity of the pore. The composition includes a plurality of liposomes and an active ingredient carried by the liposomes. The liposomes may include a cationic lipid and an amphiphilic glycerophospholipid.

The cationic lipid may comprise a C₁₋₂₀ alkane substituted with a C₁₋₂₀ acyloxy group and a tri-C₁₋₄ alkylammonium group. In some embodiments, the C₁₋₂₀ alkane is a C₁₋₅ alkane. In some other embodiments, the C₁₋₂₀ alkane is a C₁₋₃ alkane. In some embodiments, the C₁₋₂₀ alkane may comprise from one to four C₁₋₂₀ acyloxy groups. In some embodiments, the C₁₋₂₀ alkane may comprise two C₁₋₂₀ acyloxy groups. In some embodiments, the C₁₋₂₂ acyloxy groups are C₁₋₂₀ acyloxy groups. In some embodiments, the C₁₋₂₂ acyloxy groups are C₁₋₁₈ acyloxy groups.

Among the C₁₋₂₂ acyloxy groups, examples include an alkylcarbonyloxy group, an alkenylcarbonyloxy group, an alkynylcarbonyloxy group, an arylcarbonyloxy group, or a heteroarylcarbonyloxy group. In some embodiments, the C₁₋₂₂ acyloxy group is selected from the group consisting of an alkylcarbonyloxy group, an alkenylcarbonyloxy group, and an alkynylcarbonyloxy group. In some embodiments, the C₁₋₂₂ acyloxy group is an alkenylcarbonyloxy group.

The above-mentioned C₁₋₂₀ alkane may include, as a substituent, preferably one to four tri-C₁₋₆ alkylammonium groups. In some embodiments, the C₁₋₂₀ alkane may include one tri-C₁₋₆ alkylammonium group. In some embodiments, the tri-C₁₋₆ alkylammonium groups are tri-C₁₋₄ alkylammonium groups. In some embodiments, the tri-C₁₋₆ alkylammonium groups may carry one or more counter ions. Example of counter ions of the above-mentioned trialkylammonium groups include, but are not limited to, chlorine ions, bromine ions, iodine ions, fluorine ions sulfurous ions, nitrous ions, etc. In some embodiments, the counter ion is a chlorine ion, bromine ion, or iodine ion.

Specific examples of the cationic lipid include preferably 1,2-dioleoyloxy-3-(trimethylammonium) propane (DOTAP), dioctadecyldimethylammonium chloride (DODAC), N-2,3-dioleoyloxy)propyl-N,N,N-trimethylammonium (DOTMA), didodecylammonium bromide (DDAB), 1,2-dimyristoyloxypropyl-3-dimethylhydroxyethylammonium (DMRIE), and 2,3-dioleoyloxy-N-[2-(sperminecarboxamide)ethyl]-N,N-dimethyl-1-propanaminium trifluoroacetate (DOSPA). In some embodiments, the cationic lipid is DOTAP.

In some embodiments, the amphiphilic glycerophospholipid comprises a saturated fatty acid moiety and an unsaturated fatty acid moiety.

In some embodiments, the amphiphilic glycerophospholipid includes both a saturated fatty acid moiety and an unsaturated fatty acid moiety. In some embodiments, the amphiphilic glycerophospholipid is selected from the group consisting of phosphatidylcholine, phophatidylethanolamine, phosphatidylglycerol, phosphatidic acid, cardiolipin, phosphatidylserine, phosphatidylinositol, and the like. In some embodiments, the amphiphilic glycerophospholipid is phosphatidylcholine. In some embodiments, the amphiphilic glycerophospholipid is an egg-yolk phosphatidylcholine.

In some embodiments, the amphiphilic glycerophospholipid includes a saturated fatty acid moiety selected from the group consisting of C₁₂₋₂₂ saturated fatty acids and C₁₄₋₁₈ saturated fatty acids. In some embodiments, the amphiphilic glycerophospholipid comprises at least one fatty acid moiety selected from the group consisting of palmitic acid, lauric acid, myristic acid, pentadecylic acid, margaric acid, stearic acid, tuberculostearic acid, arachidic acid, and behenic acid. In some embodiments, the amphiphilic glycerophospholipid comprises at least one fatty acid moiety selected from the group consisting of palmitic acid, myristic acid, pentadecylic acid, margaric acid, and stearic acid.

Among the unsaturated fatty acid moieties, examples include C₁₄₋₂₂ unsaturated fatty acids and C₁₄₋₂₀ unsaturated fatty acids. In some embodiments, the unsaturated fatty acid moiety comprises from 1 to 6 carbon-carbon double bonds. In some embodiments, the unsaturated fatty acid moiety comprises from 1 to 4 carbon-carbon double bonds.

In some embodiments, the unsaturated fatty acid includes at least one moiety selected from the group consisting of oleic acid, myristoleic acid, pamitoleic acid, elaidic acid, vaccenic acid, gadoleic acid, erucic acid, nervonic acid, linoleic acid, α-linoleic acid, eleostearic acid, stearidonic acid, arachidonic acid, eicosapentaenoic acid, clupanodonic acid, and docosahexaenoic acid. In some embodiments, the unsaturated fatty acid includes at least one moiety selected from the group consisting of oleic acid, myristoleic acid, pamitoleic acid, elaidic acid, vaccenic acid, gadoleic acid, erucic acid, nervonic acid, linoleic acid, α-linoleic acid, eleostearic acid, stearidonic acid, and arachidonic acid.

In some embodiments, the amphiphilic glycerophospholipid includes both a saturated fatty acid moiety and an unsaturated fatty acid moiety. In some embodiments, the saturated fatty acid moiety is selected from the group consisting of palmitic acid, myristic acid, pentadecylic acid, margaric acid, and stearic acid, and the unsaturated fatty acid moiety is selected from the group consisting of oleic acid, myristoleic acid, pamitoleic acid, elaidic acid, vaccenic acid, gadoleic acid, erucic acid, nervonic acid, linoleic acid, α-linoleic acid, eleostearic acid, stearidonic acid, and arachidonic acid.

In some embodiments, the liposomes further comprise a sterol as a constituent component. The sterol may be selected from the group consisting of cholesterol, C₁₂₋₃₁ cholesteryl fatty acid, C₁₂₋₃₁ dihydrocholesteryl fatty acid, polyoxyethylene cholesteryl ether, and polyoxyethylene dihydrocholesteryl ether. In some embodiments, the sterol may be selected from the group consisting of cholesterol, cholesteryl lanoate, cholesteryl oleate, cholesteryl nonanoate, macadamia nut fatty acid, and dihydrocholesterol polyethyleneglycol ether (e.g., dihydrocholeth-30). In some embodiments, the sterol is cholesterol.

### Combination of Components

In some embodiments, as described above, the liposome includes a combination of a cationic lipid, a sterol, and an amphiphilic glycerophospholipid that includes both a saturated fatty acid and an unsaturated fatty acid. In some embodiments, as described, the liposome includes a combination of a C₁₋₂₀ alkane substituted with a C₁₋₂₂ acyloxy group and a tri-C₁₋₆ alkylammonium group; a sterol; and an amphiphilic glycerophospholipid that includes a C₁₂₋₂₂ saturated fatty acid and a C₁₄₋₂₂ unsaturated fatty acid with 1 to 6 carbon-carbon double bonds. In some embodiments, as described, the liposome includes a combination of a C₁₋₂₀ alkane substituted with two C₁₋₂₂ acyloxy groups and one tri-C₁₋₆ alkylammonium group; a sterol; and a phosphatidylcholine that includes a C₁₂₋₂₂ saturated fatty acid and a C₁₄₋₂₂ unsaturated fatty acid with 1 to 6 carbon-carbon double bonds.

In some embodiments, as described above, the liposome includes a phosphatidylcholine having, as constituent fatty acids, both of the following: at least one saturated fatty acid selected from the group consisting of 1,2-dioleoyloxy-3-(trimethylammonium)propane, sterols, palmitic acid, lauric acid, myristic acid, pentadecylic acid, margaric acid, stearic acid, tuberculostearic acid, arachidic acid, and behenic acid; and at least one unsaturated fatty acid selected from the group consisting of oleic acid, myristoleic acid, pamitoleic acid, elaidic acid, vaccenic acid, gadoleic acid, erucic acid, nervonic acid, linoleic acid, α-linoleic acid, eleostearic acid, stearidonic acid, arachidonic acid, eicosapentaenoic acid, clupanodonic acid, and docosahexaenoic acid.

In some embodiments, as described above, the liposome includes a phospatidylcholine having, as constituent fatty acids, all of the following: at least one sterol selected from the group consisting of 1,2-dioleoyloxy-3-(trimethylammonium)propane, cholesterol, cholesteryl lanoate, cholesteryl oleate, cholesteryl nonanoate, macadamia nut fatty acid, and polyoxyethylene dihydrocholesteryl ether; at least one saturated fatty acid selected from the group consisting of palmitic acid, lauric acid, myristic acid, pentadecylic acid, margaric acid, stearic acid, tuberculostearic acid, arachidic acid, and behenic acid; and at least one unsaturated fatty acid selected from the group consisting of oleic acid, myristoleic acid, pamitoleic acid, elaidic acid, vaccenic acid, gadoleic acid, erucic acid, nervonic acid, linoleic acid, α-linoleic acid, eleostearic acid, stearidonic acid, arachidonic acid, eicosapentaenoic acid, clupanodonic acid, and docosahexaenoic acid.

In some embodiments, the liposome includes 1,2-dioleoyloxy-3-(trimethylammonium)propane, cholesterol, and egg yolk phosphatidylcholine.

The liposomes may comprise an active ingredient (e.g., insulin molecules, such as insulin, insulin analogs, derivatives of insulin or insulin analogs, and the like), a cationic lipid, and an amphiphilic glycerophospholipid. The stability and iontophoretic delivery efficiency of the liposomes may depend on the ratio of the cationic lipid to the amphiphilic glycerophospholipid present in the liposomes. In some embodiments, a molar ratio of the cationic lipid to the amphiphilic glycerophospholipid ranges from about 9:1 to about 1:9. In some embodiments, a molar ratio of the cationic lipid to the amphiphilic glycerophospholipid ranges from about 3:2 to about 2:3.

In some embodiments, when the liposomes include a sterol, a molar ratio of the cationic lipid to the sterol ranges from about 19:1 to about 1:1. In some embodiments, a molar ratio of the cationic lipid to the sterol ranges from about 8:1 to about 3:1. In some embodiments, when the liposomes include a sterol, a molar ratio of the amphiphilic glycerophospholipid to the sterol ranges from about 19:1 to about 1:1. In some embodiments, a molar ratio of the amphiphilic lipid to the sterol ranges from about 8:1 to about 3:1. In some embodiments, a molar ratio of the cationic lipid to the total of the amphiphilic lipid and the sterol ranges from about 9:1 to about 1:9. In some embodiments, a molar ration of the cationic lipid to the total of the amphiphilic lipid and the sterol ranges from about 3:2 to about 2:3. In some embodiments, a molar ratio of the cationic lipid to the amphiphilic lipid and to the sterol is about 4:4:1.

In some embodiments, the average particle diameter of the liposomes is about 400 nm or greater. In some embodiments, the average particle diameter of the liposomes ranges from about 400 nm to about 1000 nm. The mean particle diameter or size of the liposomes can be determined or confirmed by, for example, a dynamic-light-scattering method, a static-light-scattering method, an electron microscope observation method, and/or an atomic force microscope observation method. In certain embodiments, the mean particle size is determined or confirmed by dynamic light scattering.

### Insulin Molecule

In some embodiments, an iontophoretic delivery composition may include one or more active ingredients in the form of a water-soluble substance, a hydrophilic substance, or a hydrophobic substance. In some embodiments, the one or more active ingredients may comprise an ionic, cationic, ionizeable, and/or neutral substance insofar as it can be carried (e.g., encapsulated) in a liposome and, for an active agent that is administered to provide a biological activity, maintains its biological activity.

In some embodiments, the active ingredient may comprise one or more insulin molecules (e.g., insulin, insulin analogs, derivatives of insulin or insulin analogs, and the like). In some embodiments, an insulin molecule is an insulin, an insulin analog, a derivative of an insulin or an insulin analog, and the like. In some embodiments, the biological activity of the one or more insulin molecules is greater than that of insulin. In some embodiments, the biological activity of the one or more insulin molecules is substantially the same as that of insulin.

In some embodiments, the insulin molecule is an insulin. In some embodiments, the insulin is a human insulin, a pig insulin, or a bovine insulin. In certain embodiments, the insulin is a human insulin. Characteristics of insulin may be found in, for example, MacPherson, J. N., Feely, J. “Insulin,” British Med. J. 300:731-736 (1990), the content of which is incorporate herein by reference.

In some embodiments, the insulin molecule is a derivative of insulin. In some embodiments, the insulin molecule is an insulin analog. In some embodiments, the insulin molecule is a derivative of an insulin analog.

In some embodiments, the insulin molecule is an insulin analog. In some embodiments, the insulin analog is an insulin wherein between one and three amino acid residues in the amino acid sequence of the insulin have been replaced by different amino acid residues. In some embodiments, the insulin analog is an insulin wherein between one and three amino acid residues have been added to the amino acid sequence of the insulin. In some embodiments, the insulin analog is an insulin wherein between one and three amino acid residues have been deleted from the amino acid sequence of the insulin. In some embodiments, the insulin analog is an ultra-fast-acting insulin analog (e.g., insulin lispro, insulin aspart, or insulin glulisine). In some embodiments, the insulin analog is a long-acting or insulin analog (e.g., insulin glargine or insulin detemir). In some embodiments, an ultra-fast-acting analog and a long-acting analog are mixed before use according to the description herein. Characteristics of insulin analogs may be found in, for example, Kurtzhals, P., et al., “Correlations of receptor binding and metabolic and mitogenic potencies of insulin analogs designed for clinical use,” Diabetes 49:999-1005 (2000); and in Daneman, D., “Type I diabetes,” Lancet 367:847-858 (2006); the contents of which are incorporated herein by reference.

In some embodiments, the insulin molecule is a derivative of an insulin or an insulin analog. In some embodiments, the derivative may be an acylated insulin or a glycosylated insulin. In some embodiments, the derivative may be a caproylated insulin, a dicaproylated insulin, a laurylated insulin, a dilaurylated insulin, a palmitoylated insulin, or a dipalmitoylated insulin. In some embodiments, the derivative may be an acylated insulin analog or a glycosylated insulin analog. In some embodiments, the derivative may be a caproylated insulin analog, a dicaproylated insulin analog, a laurylated insulin analog, a dilaurylated insulin analog, a palmitoylated insulin analog, or a dipalmitoylated insulin analog. Characteristics of such derivatives may be found in, for example, Yamamoto, A., “Biopharmaceutical study on improvement of transmucosal absorption of bioactive peptide,” Yakugaku Zasshi 121:929-948 (2001), the contents of which are incorporated herein by reference.

The disclosed liposomes and compositions comprising liposomes may be prepared in a variety of ways. In some embodiments, the disclosed liposomes, compositions, and/or formulations comprising liposomes may be prepared as described in Example 1.

### Application of Liposome Compositions

The disclosed liposome compositions and/or formulations can be advantageously utilized for intradermal administration of an insulin molecule (insulin, insulin analogs, derivatives of insulin or insulin analogs, and the like). Accordingly, in some embodiments, the disclosed liposome compositions and/or formulations may enable the stable, efficient delivery of an insulin molecule to the deep regions of a pore and intradermal tissue surrounding the pore. In some embodiments, the disclosed liposome compositions and/or formulations may be advantageously utilized to systemically deliver an insulin molecule to prevent or treat a disease. In some embodiments, the disclosed compositions and/or formulations may be utilized to systemically deliver an insulin molecule to prevent or treat diabetes. In some embodiments, the disclosed compositions maintain decreased blood glucose levels in the circulation of a biological subject over an extended period of time. In some embodiments, the disclosed compositions provide controlled or sustained release of an insulin molecule into the circulation, for example, by controlled or sustained release of a liposome enclosing an insulin molecule or by controlled or sustained release of an insulin molecule from a liposome.

In some embodiments, a method of administering an insulin molecule to a living organism by iontophoresis includes placing any of the disclosed compositions and/or formulations on the skin surface of a living body and applying an electric current to the skin. In some embodiments, the insulin molecule is carried (e.g., enclosed, encapsulated, and the like) in the liposomes in the composition and administered to a living organism through, for example, a skin pore.

In some embodiments, the disclosed liposome compositions and/or formulations may be directly placed on the skin surface, or may be part of an electrode structure of an iontophoresis device in which the composition is held, stored, or carried. In use, the iontophoresis device is placed on a skin surface and electric current is applied to an electrode structure holding, storing, or carrying a composition of liposomes encapsulating an insulin molecule, thereby administering the insulin molecule iontophoretically.

For cationic liposomes, the anode of an iontophoresis device is supplied with an electric current. In some embodiments, the electric current supplied by the iontophoretic device and applied to the liposomes ranges from about 0.1 mA/cm² to about 0.6 mA/cm². In some embodiments, the electric current supplied by the iontophoretic device ranges from about 0.3 mA/cm² to about 0.5 mA/cm². In some embodiments, the electric current supplied by the iontophoretic device is about 0.45 mA/cm². In some embodiments, a period of time for applying electric current to the electrode structure ranges from about 5 minutes to about 2 hours; in some embodiments, from about 10 minutes to about 1.5 hours; and, in some further embodiments, about 1 hour.

In some embodiments, the living organism includes any mammal, such as, for example, a rat, a human, a guinea pig, a rabbit, a mouse, and a pig. In some embodiments, the living organism is a human.

### Electrode Assembly and Device for Iontophoresis

In some embodiments, the disclosed compositions and/or formulations may be held in, stored, carried, or be part of, an electrode structure suitable for iontophoretic delivery of the compositions and/or formulations. In some embodiments, the electrode structure for administering an active ingredient (e.g., an insulin molecule, such as an insulin, an insulin analog, a derivative of an insulin or an insulin analog, and the like) to a living body via iontophoresis comprises one or more of the disclosed compositions and/or formulations. In some embodiments, the liposomes take the form of cationic liposomes, and the electrode structure is configured such that the anode side of the electrode structure is configured to transdermally deliver the composition including the liposomes, when current and/or a potential is applied to the electrode structure.

In some embodiments, the electrode structure includes at least a positive electrode and an insulin molecule holding portion capable of holding any of the disclosed compositions and/or formulations.

In some embodiments, the insulin molecule holding portion may be directly disposed on the front surface of the positive electrode and other components such as, for example, an ion exchange membrane, may be disposed between the positive electrode and the insulin molecule holding portion insofar as the administration of liposomes by iontophoresis is not substantially hindered.

In some embodiments, the electrode structure comprises at least a positive electrode, an electrolyte holding portion for holding electrolyte disposed on the front surface of the positive electrode, an anion exchange membrane disposed on the front surface of the electrolyte holding portion, and an insulin molecule holding portion for holding any of the disclosed compositions and/or formulations. In some embodiments, a cation exchange membrane may be disposed on the front surface of the above-mentioned active ingredient holding portion.

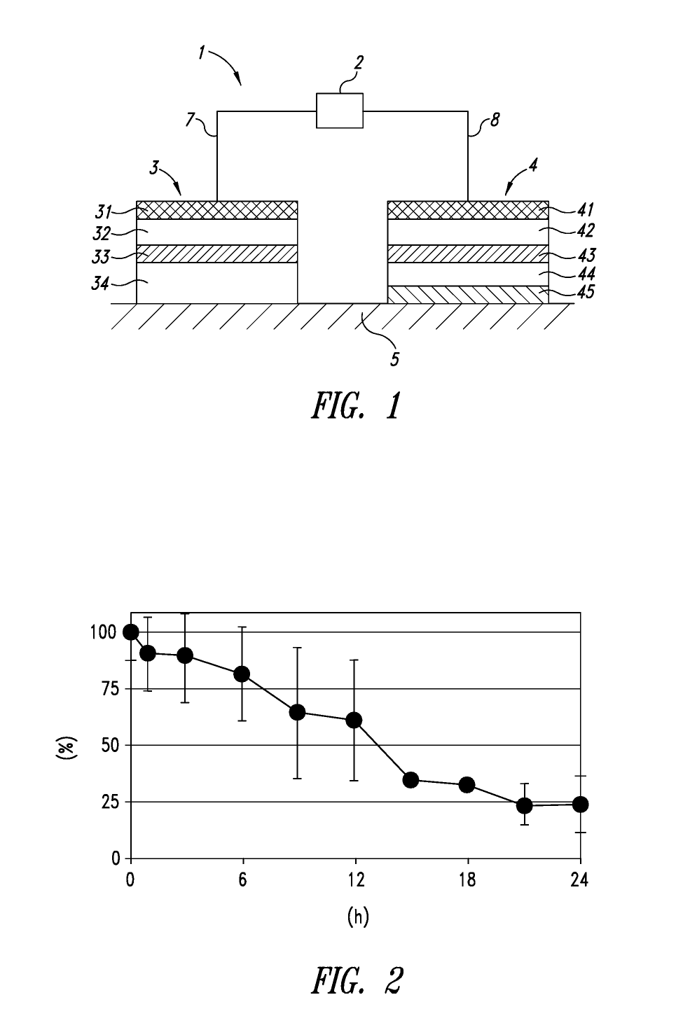

As shown in FIG. 1, in some embodiments, an iontophoresis device 1 may include any of the disclosed electrode structures, or any other structure suitable for iontophoretic delivery of the active ingredient or any of the disclosed compositions and/or formulations. In some embodiments, the iontophoresis device 1 may include at least a power supply 2, an electrode structure 3 connected to the power supply 2, and a counter electrode structure 4. The electrode structure 3 may serve to hold any of the disclosed compositions and/or formulations. The structure of the counter electrode 4 is not limited insofar as the administration of liposomes by iontophoresis is not substantially hindered. For example, the counter electrode 4 may include a negative electrode 4, an electrolyte holding portion 42 for holding electrolyte disposed on the front surface of the negative electrode 4, and an ion exchange membrane disposed on the front surface of the electrolyte holding portion 42. The above-mentioned ion exchange membrane may be an anion exchange membrane or a cation exchange membrane, and preferable is an anion exchange membrane.

An example of an electrode structure 3 and an iontophoresis device 1 is illustrated in FIG. 1. Further examples include those disclosed in, for example, International Publication WO 03/037425 A1.

Liposomes may migrate to a side opposite to the positive electrode due to an electric field resulting from applying an electric current, and may be efficiently emitted from the electrode structure. In some embodiments, a method of operating an iontophoresis device includes placing the electrode structure 3, comprising a plurality of liposomes carrying an active ingredient, and the counter electrode structure 4 on the skin surface of a living body 5, and applying a sufficient electric current to the iontophoresis device 1 so as to emit a substantial amount of the liposomes held in active ingredient holding portion 34 of the electrode structure.

In the above-mentioned iontophoresis device 1, the active ingredient holding portion 34 or the electrolyte holding portion 32 may be formed of a reservoir (electrode chamber) which is, for example, formed of acrylic and is filled with any of the disclosed compositions and/or formulations, or with an electrolyte, and may be formed of a thin film body having properties of holding and/or retaining the disclosed compositions and/or formulations, or electrolyte. With respect to the thin film body, the same material can be used in the active ingredient holding portion 34 and the electrolyte holding portion 32.

The disclosed methods and device may employ any suitable electrolyte. In some embodiments, a suitable electrolyte can be selected based on the conditions and properties of the active ingredient. However, electrolytes that adversely affect the skin of a living body due to an electrode reaction should be avoided. Suitable electrolytes include organic acids and salts thereof. Those organic acids and salts thereof that take part or exist in a metabolic cycle of a living body are generally preferable from the viewpoint of non-toxicity. For example, suitable electrolytes include lactic acid and fumaric acid. In some embodiments, the suitable electrolyte is a one to one (1:1) aqueous solution of 1M lactic acid and 1M sodium fumarate.

It is important that the thin film body forming the active ingredient holding unit have the ability to absorb and/or retain any of the disclosed compositions, formulations, and/or electrolyte and have the ability to migrate ionized liposomes absorbed in and/or retained by the thin film body under predetermined electric field conditions to the skin side (ion transportation ability, ion electrical conductivity). Exemplary materials having both favorable absorbance and retaining properties and favorable ion transportation ability include a hydrogel body of an acrylic resin (acrylic hydrogel membrane), a segmented polyurethane-based gel membrane, an ion conductive porous sheet for the formation of a gel-like solid electrolyte (e.g., a porous polymer which includes an acrylonitrile copolymer containing acrylonitrile in an amount of 50 mol % or more, preferably 70 to 98 mol % or more, and having a void ratio of 20 to 80%, as disclosed in Japanese Patent Application No. 11-273452 A), and the like. When adding (e.g., impregnating, permeating, loading, and the like) any of the disclosed compositions and/or formulations to the above-mentioned active ingredient holding unit 34 the impregnation and/or permeation degree (100×(W−D)/D [%], where D refers to dry weight and W refers to weight after impregnation) is preferably from about 30% to about 40%.

The conditions for loading the active ingredient holding portion 34 or the electrolyte solution holding portion 32 with any of the disclosed compositions, formulations, and/or electrolytes are suitably determined according to the amount of electrolyte or ionic drug to be loaded, the absorption rate, etc. In some embodiments, the loading of the active ingredient holding portion is performed at, for example, 40° C. for 30 minutes.

In some embodiments, the inert electrode may be composed of, for example, a conductive material such as carbon or platinum and may be used as the electrode of the electrode assembly.

In some embodiments, a cation exchange membrane and an anion exchange membrane can be used in combination in the electrode assembly. Examples of cation exchange membranes include NEOSEPTA's manufactured by Tokuyama Soda, Co., Inc. (CM-1, CM-2, CMX, CMS, CMB, and CLE 04-2). Examples of anion exchange membranes include NEOSEPTA's manufactured by Tokuyama Soda, Co., Inc. (AM-1, AM-3, AMX, AHA, ACH, ACS, ALE 04-2, and AIP-21). Further examples of the membranes include a cation exchange membrane obtained by partially or entirely filling the pore portions of a porous film with an ion exchange resin having a cation exchange function and an anion exchange membrane obtained by partially or entirely filling the pore portions of a porous film with an ion exchange resin having an anion exchange function.

Details about the respective components and the like described above may be found in, for example, International Patent WO 03/037425A1 by the applicant of the present disclosure, the entire contents of which are incorporated into the present disclosure.

The various embodiments described herein are further illustrated by the following non-limiting examples.

### EXAMPLES

### Example 1

First, cationic lipid, amphiphilic glycerophospholipid, and optionally sterol or the like are mixed in desired ratios in an organic solvent such as CHCl₃ to obtain a suspension. The suspension is distilled under reduced pressure, and the addition of an organic solvent and distillation under reduced pressure are repeated, to yield a lipid film. Next, to the lipid film, a buffer such as 10 mM to 50 mM HEPES (4-[2-hydroxyethyl]-1-piperazineethanesulfonic acid) or the like and a desired amount of active ingredient are added. The resulting mixture is left standing at room temperature for 10 minutes for hydration, followed by sonication. The sonication is performed in a sonicator, for example, at room temperature at 85 W for 1 minute, but the conditions are not limited thereto. The mixture is treated using a membrane filter, extruder, etc., to adjust the particle diameter, thereby obtaining liposomes. The liposomes are further mixed with a pharmacologically acceptable carrier and the like, thereby obtaining a composition and/or formulation of liposomes.

A number of pharmacologically acceptable carriers and excipients may be used with the disclosed composition and/or formulations, and methods insofar as the administration of liposomes by iontophoresis is not substantially hindered. For example, surfactants, lubricants, dispersants, buffers such as HEPES, additives such as preservatives, solubilizing agents, antiseptics, stabilizing agents, antioxidants, colorants, may be included. The liposome composition can be formed into a suitable dosage form as desired, insofar as the administration of liposomes by iontophoresis in not substantially hindered.

In some embodiments, the composition of liposomes is formed into a solution or suspension with HEPES buffer and/or any of the disclosed electrolytes. The disclosed composition and methods can be applied to various uses according to types and properties of an active ingredient to be enclosed in liposomes.

### Example 2

### Preparation of Liposome Formulation

First, a liposome formulation for iontophoresis was prepared by encapsulating insulin (MP Biochemicals, Inc.) in a liposome comprising a cationic lipid DOTAP with a stable lipid membrane composition capable of being used in iontophoresis by the following method.

A solution of 10 mM DOTAP (Avanti Polar Lipids, Inc.) in CHCl₃ (250 μL), a solution of 10 mM cholesterol (hereinafter referred to as “Chol”; Avanti Polar Lipids, Inc.) in CHCl₃ (125 μL), and a solution of 10 mM of egg yolk phosphatidylcholine (hereinafter referred to as “EPC”; NOF Corporation) in CHCl₃ (250 μL) were mixed, and 500 μL of CHCl₃ were added to the mixture, to yield a suspension (molar ratio DOTAP:EPC:Chol=5:5:1.25). After removal of the solvent from the suspension by distillation, under reduced pressure using an evaporator, 400 μL of CHCl₃ were added to the remainder, and the solvent was again removed from the mixture by distillation under reduced pressure, whereby a lipid film was obtained. To the lipid film were added 1 mL of 10 mM HEPES buffer and 0.5 mL of a solution of 2.4 mg (corresponding to 29 IU/mg))/mL of insulin in a 10 mM phosphate buffer (pH 7.4). The resultant mixture was allowed to stand at room temperature for 10 minutes to achieve hydration and was then subjected to sonication (85 W, room temperature, 1 minute; AU-25C ultrasonic cleaner, Aiwa Ika Kogyo K. K.). The sonicated mixture was then subjected to six freeze-thaw cycles, after which 1.5 mL of 200 mM phosphate buffer was added. The mixture was then treated with an extruder (Mini-Extruder, Avanti Polar Lipids, Inc.) using a PC membrane with a pore size of 1,000 nm (Nuclepore Track-Etch Membrane, manufactured by Whatman), whereby a liposome suspension was obtained, having an encapsulated insulin concentration of 1.2 mg/M. Unencapsulated insulin was removed from the liposome suspension by centrifugation at 65,000×g, for 30 minutes, at 4° C. The supernatant was decanted and the resulting liposome pellet was resuspended in 0.35 mL of 10 mM HEPES buffer, pH 7.4. The mean particle diameter of the resulting liposome formulation was determined by dynamic light scattering (Zetasizer Nano-ZS, Sysmex Corp.) to be in a range of about 300 to 500 nm.

The amount of insulin encapsulated in each liposome preparation was determined by solubilizing the liposomes in a surfactant solution and quantifying protein content with a BCA (bicinchoninic acid) protein quantification kit (Pierce Chemical Co., USA), using a calibration curve prepared with bovine serum albumin. The mean yield of insulin encapsulated in the liposome formulations was determined to be about 65% [=(weight of insulin contained in the final liposome preparation)/(weight of insulin added in preparing the liposomes)×100].

The concentration of insulin-encapsulated liposomes in the liposome preparation was adjusted to 2.0 mg/mL with 10 mM HEPES buffer, pH 7.4, and used in a transdermal administration test (Example 3).

### Example 3

### Transdermal Administration Test

Streptozotocin (STZ, 150 mg/kg body weight) was administered to SD rats (male; 9-week-old; CLEA Japan, Inc.; mean body weight 235 g and mean normal blood glucose level about 120 mg/dL; n=5) to induce type I diabetes. After administration of STZ, the blood glucose levels of the SD rats ranged from about 300 mg/dL to about 400 mg/dL. After treatment of the SD rats with STZ, the liposome formulation of Example 2 was transdermally administered to the back of each rat by iontophoresis using the following protocol.

First, anesthesia (1 mL of Nembutal (50 mg/ml) per 1 kg of a body weight) was administered to each SD rat, and the hair on the back of each rat was shaved. Next, as shown in FIG. 1, an iontophoresis device 1 including an electric power source device 2, a working electrode assembly 3, and a counter electrode assembly 4 was placed on a biological surface, such as, for example, exposed skin 5. 100 μL of the above liposome suspension was applied in advance to a surface where the exposed skin 5 and the working electrode assembly 3 contacted each other.

The working electrode assembly 3, of iontophoresis device 1, included, as previously disclosed: a positive electrode 31; an electrolyte solution holding portion 32 for holding 1 mL of an electrolyte solution (physiological saline), the electrolyte solution holding portion 32 being placed on the front surface of the positive electrode 31; an anion exchange membrane 33; and an insulin holding portion 34 for holding 200 μL of the liposome suspension, the insulin holding portion 34 being placed on the front surface of the anion exchange membrane 33.

The counter electrode assembly 4 included: a negative electrode 41; an electrolyte solution holding portion 42 for holding 1 mL of an electrolyte solution, the electrolyte solution holding portion 42 being placed on the front surface of the negative electrode 41; a cation exchange membrane 43; an electrolyte solution holding portion 44 for holding 800 μL of a physiological saline, the electrolyte solution holding portion 44 being placed on the front surface of the cation exchange membrane 43; and an anion exchange membrane 45 placed on the front surface of the electrolyte solution holding portion 44. In addition, ion exchange membranes stored in physiological saline in advance were used as the above anion exchange membranes 33 and 45 (ALE 04-2, Tokuyama Soda, Co., Inc.), and the cation exchange membrane 43 (CLE 04-2, Tokuyama Soda, Co., Inc.).

Next, the liposome formulation was administered to a number of rats with the iontophoresis device 1 shown in FIG. 1 using a current of about 1.14 mA (0.45 mA/cm²) for about 20 minutes.

Changes in blood glucose levels of the STZ-treated SD rats over time after iontophoretic administration of the liposome formulation were measured using an automated blood sampling device (blood sampling device DR-II, Eicom). The blood glucose levels were measured over a period of 24 hours, during which the SD rats were fasting.

The changes over time in the blood glucose levels of the SD rats are shown in FIG. 2. The blood glucose level at each time point is represented in FIG. 2 as mean value±standard deviation. Each time point represents the lapsed time since initiating administration of the insulin-encapsulated liposome formulation. Under the iontophoretic conditions described above (1.14 mA (0.45 mA/cm²) for 20 minutes), a decrease in mean blood glucose level of the SD rats was observed beginning at about the 9 hour time point. After about 15 hours, the mean blood glucose reached a level about 25% of the level at the beginning of administration.

The results show that administration of an insulin-encapsulated liposome formulation iontophoretically through a skin as described herein can efficiently decrease blood glucose levels.

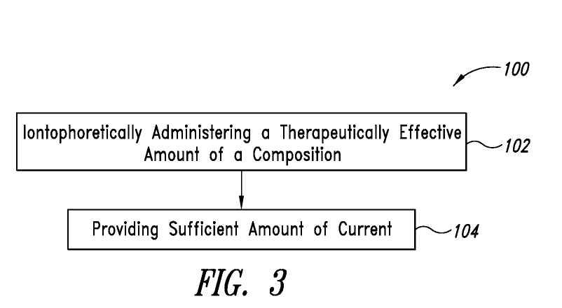

FIG. 3 shows an exemplary method 100 for preventing or treating a condition or a disease associated with increased blood glucose levels in a biological subject.

At 102, the method 100 includes iontophoretically administering to the biological subject in need of such treatment a therapeutically effective amount of a composition comprising a plurality of liposomes comprising a cationic lipid, an amphiphilic glycerophospholipid having a saturated fatty acid moiety and an unsaturated fatty acid moiety, and at least one insulin molecule, wherein the at least one insulin molecule includes more than one insulin molecule. In some embodiments, the at least one insulin molecule is carried by the plurality of liposomes. In some embodiments, the at least one insulin molecule is selected from insulin, insulin analogs, derivatives of insulin, or derivatives of insulin analogs.

In some embodiments, the cationic lipid is present in a molar ratio of the cationic lipid to the amphiphilic glycerophospholipid of about 9:1 to about 1:9.

In some embodiments, iontophoretically administering to the biological subject in need of such treatment the therapeutically effective amount of a composition comprises providing a current ranging from about 0.1 mA/cm² to about 0.6 mA/cm² for a pre-selected period of time. In some embodiments, iontophoretically administering to the biological subject in need of such treatment the therapeutically effective amount of a composition comprises providing a current ranging from about 0.3 mA/cm² to about 0.5 mA/cm² for a pre-selected period of time. In some embodiments, iontophoretically administering to the biological subject in need of such treatment the therapeutically effective amount of a composition comprises providing a current of about 0.45 mA/cm² for a pre-selected period of time.

At 104, the method 100 may further include providing a sufficient amount of current to deliver a therapeutically effective amount of the composition to the biological subject.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A composition, comprising:
a plurality of liposomes comprising:
a cationic lipid, and
an amphiphilic glycerophospholipid having a saturated fatty acid moiety and an unsaturated fatty acid moiety; and
at least one insulin molecule;
wherein the at least one insulin molecule is enclosed within a liposome; and
wherein the composition is suitable for iontophoretic delivery of the at least one insulin molecule to a biological subject.

2. The composition according to claim 1 wherein the at least one insulin molecule is selected from the group consisting of an insulin, an insulin analog, a derivative of an insulin, and a derivative of an insulin analog.

3. The composition according to claim 2 wherein the at least one insulin analog is an ultra-fast-acting insulin analog or a long-acting insulin analog.

4. The composition according to claim 1 wherein the composition provides for controlled or sustained release of an insulin molecule.

5. The composition according to claim 1 wherein the cationic lipid comprises a C₁₋₂₀ alkane substituted with a C₁₋₂₂ acyloxy group and a tri-C₁₋₆ alkylammonium group.

6. The composition according to claim 1 wherein the cationic lipid comprises a C₁₋₂₀ alkane substituted with at least two C₁₋₂₂ acyloxy groups and at least one tri-C₁₋₆ alkylammonium group.

7. The composition according to claim 1 wherein the cationic lipid comprises 1,2-dioleoyloxy-3-(trimethylammonium)propane.

8. The composition according to claim 1 wherein the amphiphilic glycerophospholipid comprises phosphatidylcholine or an egg yolk phosphatidylcholine.

9. The composition according to claim 1 wherein the saturated fatty acid moiety is a C₁₂₋₂₂ saturated fatty acid.

10. The composition according to claim 1 wherein the saturated fatty acid moiety is selected from the group consisting of palmitic acid, lauric acid, myristic acid, pentadecylic acid, margaric acid, stearic acid, tuberculostearic acid, arachidic acid, and behenic acid.

11. The composition according to claim 1 wherein the saturated fatty acid moiety comprises 1, 2, 3, 4, 5 or 6 carbon-carbon unsaturated double bonds.

12. The composition according to claim 1 wherein the unsaturated fatty acid moiety is a C₁₄₋₂₂ unsaturated fatty acid.

13. The composition according to claim 1 wherein the unsaturated fatty acid moiety is selected from the group consisting of oleic acid, myristoleic acid, palmitoleic acid, elaidic acid, vaccenic acid, gadoleic acid, erucic acid, nervonic acid, linoleic acid, α-linoleic acid, eleostearic acid, stearidonic acid, arachidonic acid, eicosapentaenoic acid, clupanodonic acid, and docosahexaenoic acid.

14. The composition according to claim 1 wherein a molar ratio of the cationic lipid to the amphiphilic glycerophospholipid is from about 9:1 to about 1:9.

15. The composition according to claim 1 wherein a molar ratio of the cationic lipid to the amphiphilic glycerophospholipid is from about 3:2 to about 2:3.

16. The composition according to claim 1 wherein the liposome further comprises a sterol.

17. The composition according to claim 16 wherein the sterol is selected from the group consisting of cholesterol, C₁₂₋₃₁ cholesteryl fatty acid, C₁₂₋₃₁ dihydrocholesteryl fatty acid, polyoxyethylene cholesteryl ether, and polyoxyethylene dihydrocholesteryl ether.

18. The composition according to claim 16 wherein the sterol is selected from the group consisting of cholesterol, cholesteryl lanolate, cholesteryl oleate, cholesteryl nonanate, macadamia nut fatty acid cholesteryl, and polyoxyethylene dihydrocholesteryl ether.

19. The composition according to claim 16 wherein the sterol is cholesterol.

20. The composition according to claim 16 wherein a molar ratio of the cationic lipid to the sterol is from about 19:1 to about 1:1.

21. The composition according to claim 16 wherein a molar ratio of the amphiphilic glycerophospholipid to the sterol is from about 19:1 to about 1:1.

22. The composition according to claim 16 wherein a molar ratio of the cationic lipid to the total of the amphiphilic glycerophospholipid and the sterol is from about 9:1 to about 1:9.

23. The composition according to claim 16 wherein a molar ratio of the cationic lipid, to the amphiphilic glycerophospholipid, and to the sterol is about 4:4:1.

24. The composition according to claim 1 wherein an average particle diameter of the liposomes is about 400 nm or greater.

25. The composition according to claim 1 wherein an average particle diameter of the liposome ranges from about 400 nm to about 1000 nm.

26. A method for treating or preventing a condition or a disease associated with increased blood glucose levels in a biological subject, comprising:
iontophoretically administering to the biological subject in need of such treatment a therapeutically effective amount of a composition comprising a plurality of liposomes comprising a cationic lipid, an amphiphilic glycerophospholipid having a saturated fatty acid moiety and an unsaturated fatty acid moiety, and at least one insulin molecule, the at least one insulin molecule being carried by the plurality of liposomes, the cationic lipid present in a molar ratio of the cationic lipid to the amphiphilic glycerophospholipid of about 9:1 to about 1:9, and the liposome having a mean particle diameter of about 400 nm to about 1000 nm.

27. The method of claim 26 wherein iontophoretically administering to the biological subject in need of such treatment the therapeutically effective amount of a composition comprises providing a current ranging from about 0.1 mA/cm² to about 0.6 mA/cm² for a pre-selected period of time.

28. The method of claim 26 wherein iontophoretically administering to the biological subject in need of such treatment the therapeutically effective amount of a composition comprises providing a current ranging from about 0.3 mA/cm² to about 0.5 mA/cm² for a pre-selected period of time.

29. The method of claim 26 wherein iontophoretically administering to the biological subject in need of such treatment the therapeutically effective amount of a composition comprises providing a current of about 0.45 mA/cm² for a pre-selected period of time.

30. The method of claim 26 wherein the condition or disease associated with increased blood glucose levels is diabetes mellitus.

31. The method of claim 30 wherein the diabetes mellitus is diabetes mellitus type 1.
